(19) United States
(12) Patent Application Publication
(10) Pub. No.: US 2003/0021817 A1
(43) Pub. Date: Jan. 30, 2003

(54) Iridescent cosmetic composition and uses thereof

(76) Inventors: Laurence Arnaud-Sebillotte, Les Roses (FR); Veronique Guillou, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/182,398

(22) PCT Filed: Jan. 14, 2002
(86) PCT No.: PCT/FR02/00127

(30) Foreign Application Priority Data
Jan. 18, 2001 (FR) 01-00684

(51) Int. Cl. A61K 7/021; A61K 7/00

(52) U.S. Cl. 424/401; 424/63

(57) ABSTRACT

The invention relates to a topically-applied iridescent composition comprising at least one cationic or amphoteric polymer and an aqueous dispersion of polymer particles, said particles having an average size of between 50 and 300 nm. The inventive composition has a very attractive appearance owing to the iridescent color thereof. The invention also relates to uses for said composition in the cosmetic field, particularly for treating, protecting, caring for, removing make-up from and/or cleansing the skin, lips and/or hair, and/or for applying make-up to the skin and/or lips.

The subject of the invention is an iridescent composition for topical application, comprising at least one cationic or amphoteric polymer and an aqueous dispersion of particles of polymer, the said particles having a mean size in numerical terms ranging from about 50 to 300 nm, and the uses of the said composition, in particular for the treatment, protection, care, removal of make-up from and/or cleansing of the skin, the lips and/or the hair, and/or for the application of make-up to the skin and/or the lips.

Cosmetic or dermatological products in gel form, for the care of or cleansing of/removal of make-up from the face, the body or the hair, are provided in general in the form of transparent or translucent homogeneous gels. Moreover, they may contain cationic polymers with the aim either of promoting the deposition of active agents on the skin or the hair, or of imparting smoothness on the composition.

To make them more attractive, they can be coloured by adding various colouring agents thereto. These colouring agents may be, for example, pigments such as lacquers, inorganic pigments or pearlescent pigments, or soluble colorants. To obtain iridescent coloured effects, pearlescent pigments of varying colours may be used. However, most often, the iridescent effect obtained with these pearlescent pigments is fairly weak, and, in addition, these pigments are difficult to disperse and to maintain in suspension, in particular in fairly fluid compositions. The incorporation of such pigments is therefore delicate and the reproducibility of the effect obtained is not certain. Another means of making these gels more aesthetic is to include liquid crystals, but industrial equipment, which is expensive, is required to homogeneously introduce them, and, moreover, they tend to be sticky.

It has therefore been sought to design products, in particular conditioning products for the skin and/or the hair, containing cationic or amphoteric polymers, having an iridescent effect without incorporating pearlescent pigments.

The applicant has found, surprisingly, that polymers, in the form of monodisperse particles in dispersion (in emulsion or in latex form) allows the production of products with an iridescent appearance, which are very attractive for consumers without having the technical difficulties of suspension of the particles.

Indeed, compositions containing a colouring system, which is obtained from colloidal crystalline networks in the medium, giving an iridescent colour without the addition of pigments or colorants, are known from the document WO-A-00/47167. However, to obtain such an effect, the medium has to have a relatively low ionic strength and a conductance of less than 2.5 μΩ⁻¹ (the conductance of a composition is the product of the conductivity times the measuring cell constant), which limits the addition of ionic additives such as ionic polymers and certain active agents.

The need therefore remains for an iridescent composition which can retain this iridescent character regardless of the conductance and the ionic strength of its constituent medium.

The applicant has found that it was possible to incorporate raw materials having an ionic strength markedly greater than 2.5 μΩ⁻¹, such as, for example, cationic or amphoteric polymers having charge densities of between 0.9 and 4 meq/g, without damaging the irisation using aqueous dispersions of particles of polymers, in which the mean size of the particles in numerical terms ranges from about 50 to 300 nm, this size being that of the original particles before any mixing with other constituents which may cause modification of the size, for example their swelling.

Accordingly, the subject of the present invention is an iridescent composition for topical application, comprising at least one polymer chosen from cationic polymers and amphoteric polymers, and monodisperse particles of polymer in aqueous dispersion, the said particles having a mean size in numerical terms ranging from 50 to 300 nm.

“Mean size in numerical terms” refers to the mean diameter in numerical terms of the preferably spherical particles of polymer of the dispersion, this size being the original size of the particles before they are mixed with other constituents. Indeed, the size of the particles may be different before and after their incorporation into a composition, because, as described below, the particles may consist of polymer comprising a monomer which is soluble or which swells in alkaline media, such that the particles of polymer may swell in water or alkaline media after incorporation into the composition for topical application.

The size of the particles is measured by means of the Brookhaven 90Plus particle size analyser. The measurements are carried out at 90°.

The composition of the invention is characterized by its iridescent effect and it exhibits the advantage of having this iridescent effect regardless of the value of its conductance (expressed as Ohm⁻¹ or as Siemens)

Macroscopically, the iridescent effect results in variations in colour which are perceived by an observer who would be moving around the surface of the product illuminated by a fixed light beam. By contrast, a product is not iridescent when the observer does not see a change in colour during their movement.

The iridescent effect may be measured by means of a goniometer, the principle of which is to measure the colour by varying the geometric conditions of observations (angles of illumination and of detection of the reflected light). The method of measurement used here and described in greater detail below is inspired by that described in “Color effects from thin film designs” by Roger Phillips, Mike Nofi and Robert Slusser (Flex Products Inc 2793 Nothpoint Parkway Santa Rosa Calif. 95407).

Method of Measurement:

The samples, at the temperature of 23° C., fill a vessel 5 mm in height. Their top surface is placed in the plane of measurement of a spectrophotogoniometer. The spectra are measured as a reflexion at 400-760 nm in steps of 5 nm. The spectra are recorded at an angle of illumination set at 55° and at angles of detection placed successively at the following values: 90° (scattering), 100, 110, 120, 130 and 1400. This path simulates the variations perceived by an observer who would be moving around the surface of the product illuminated by a fixed light beam.

The different spectra thus obtained are processed in order to obtain the calorimetric parameters L*a*b* in the CIELAB space (reference: ceramic bore MINOLTA Number 20231050 at the illumination D65 Y=94.1, x=0.3157, y=0.3331). The colour path is obtained by joining in the plane of abscissa a* and of ordinate b*, the coordinates of the calorimetric values calculated. a* varies from green to red and b* varies from blue to yellow. When a* is negative, the colour possesses a dominant green; when it is positive, the colour possesses a dominant red. When b* is negative, the colour possesses a dominant blue; when it is positive, the colour possesses a dominant yellow. The calorimetric difference is equal to the square root of the sum of the squares of the differences in the values a and b relative to the first measurement (detection angle of 90 degrees, a measurement close to that obtained with a conventional calorimeter such as the chromameter MINOLTA CR300).

Thus, a composition has an iridescent appearance when the calorimetric difference for this composition or for the dispersion of particles of polymer which it contains is greater than 2, and preferably it varies from 2 to 100, preferably from 3 to 60 for an angle of illumination of 55° and an angle of detection of between 100 and 140° as described above. The incident light is characterized by a beam having a diameter of 8 mm, an angular resolution of 1.3 degrees, a wavelength of 250 to 800 nm and a 1 nm type resolution.

A product which does not possess an iridescent effect is characterized, for example, by differences varying from 0.1 to 1 under the same measurement conditions.

The composition of the invention is also characterized by its turbidity, that is to say its opacity. The turbidity measures the opacity of a product. The NTU (Nephelometric Turbidity Units) are the units of measurement of the turbidity of a composition. The measurement of turbidity may be carried out, for example, with a turbidimeter model 2100P from the company HACH, the tubes used for the measurement being identified by the references AR397A cat 24347-06. The measurements are carried out at room temperature (20° C. to 25° C.). The higher the turbidity, the greater the opacity of the product. The composition of the invention is generally translucent to opaque and it preferably exhibits a turbidity of greater than 100.

Moreover, the composition according to the invention generally has a viscosity ranging from 1 poise to 100 poises (0.1 Pa.s to 10 Pa.s), preferably from 4 to 70 poises (0.4 to 7 Pa.s) and more preferably from 5 to 30 poises (0.5 to 3 Pa.s), this viscosity being measured at about 25° C. with a Rheomat 180 apparatus using a 2, 3, 4 or 5 rotor depending on the viscosity range, at 200 s⁻¹.

The composition of the invention is a composition for topical, and in particular cosmetic, use, and, as such, it contains a physiologically acceptable medium, that is to say a medium compatible with the skin, the hair, the nails and/or the mucous membranes (lips). In addition, it preferably has a pH compatible with the skin, that is to say preferably ranging from 3 to 8 and, even better, from 5 to 7. This pH value may depend on the type of polymer used.

The polymer particles used in the composition of the invention should be monodisperse, that is to say that the results of measurements of the diameter of the particles are statistically distributed around a mean and according to a single Gaussian curve. The variation relative to the mean should not exceed 10% per 100% of particles. This means that the particles practically all have the same size. This size ranges from 50 to 300 nm, preferably from 90 to 230 nm and even better from 100 to 200 nm. The size of the particles is measured by means of the Brookhaven 90Plus particle size analyser. The measurements are carried out at 900.

The particles consist of polymers. They become suspended or dispersed in water, in latex or emulsion form. Latexes are aqueous dispersions of particles of polymers, as described in “An introduction to polymer colloids” by F. CANDAU and R. H. OTTEWILL, Kluwer Academic Publishers, March 1989. The term polymer is understood here to mean both homopolymers obtained from a single type of monomer and copolymers obtained from several types (two or more) of monomers. These polymers may be either associative polymers (that is to say possessing a hydrophobic part and a hydrophilic part) or nonassociative polymers (that is to say hydrophilic or water-soluble). They are dispersible in water and may exhibit swelling in alkaline media (or alkaline-swelling) or not. The polymers may be of any type: nonionic, anionic, cationic, zwitterionic or amphoteric.

The particles which can be used in the composition of the invention preferably consist of ionic polymers and even better of anionic polymers. These polymers are dispersible in water and preferably exhibit alkaline-swelling. The preferred polymers possess at least one monomer soluble in alkaline media, such as monomers of acrylic, methacrylic, vinylacetic, maleic, crotonic and itaconic acids. They may contain another monomer such as styrene, butadiene, ethylene, acrylonitrile, chloroprene, vinylidene chloride, isoprene, isobutylene, vinyl chloride, and esters of acrylic, methacrylic or vinylacetic, maleic, crotonic and itaconic acids and mixtures thereof.

Thus, the polymer particles may be chosen, for example, from the particles consisting of the following anionic polymers:

1) homopolymers of acrylic acid, such as those of the dispersions (in aqueous emulsion) marketed under the names MIRACARE XC 96/36, MIRACARE XC 96/37 and MIRACARE XC 96/52 (acrylic nanolatex) by the company RHODIA CHIMIE.

2) copolymers of acrylic acid and of other monomers, such as those of the dispersions marketed under the names NEOCRYL PD-723-B (particle size: 114 nm), NEOCRYL XK 90 (particle size: 109 nm), NEOCRYL XK 53 (particle size: 96 nm) by the company AVECIA RESINS; ECOCRYL VS 301 (particle size: 150 nm) by the company ATOCHEM; NEOCRYL XK-75 (particle size: 102 nm) (aqueous emulsion of an acrylic acid/butyl methacrylate/methyl methacrylate/butyl acrylate copolymer) by the company AVECIA RESINS; MIRACARE XC 97-8 to 13 (acrylic/methyl methacrylate/butyl acrylate/methacrylic acid copolymer and derivatives in nonionic aqueous nanoemulsion) by the company RHODIA CHIMIE, ACRYSOL 33 (or ACULYN 33) (acrylic acid/ethyl acrylate copolymer) (particle size: 110 nm) by the company ROHM & HAAS.

3) styrene derivatives, such as those of the dispersions marketed under the name SETALUX 6801 AQ 24 (styrene/butyl acrylate/methyl methacrylate/methacrylic acid hybrid copolymer) (particle size: 186 nm) by the company AKZO NOBEL; under the name RHODOPAS GS 125 (particle size: 170 nm) by the company RHONE POULENC, under the names NEOCRYL XK 61 (anionic acrylic/styrene copolymer) (particle size: 76 nm), NEOCRYL A 1079 (styrene/acrylate copolymer) (particle size: 115 nm) by the company AVECIA RESINS; under the name RK-3-59A-PMN (mixture of two styrene/acrylic polymers (nucleus/envelope)) (particle size: 116 nm) by the company ROHM-HAAS, under the name DOW LATEX 432 (styrene/acrylate copolymer) (particle size: 145 nm) by the company LAMBERT-RIVIERE, under the name SCX 8060 (particle size: 116 nm) by the company JOHNSON POLYMER France.

4) fluorinated derivatives, such as those of the dispersions marketed under the references LUMIFLON E-3029 (anionic aqueous dispersion containing 50% of hydroxylated fluorinated polymer) (particle size: 100 nm) and LUMIFLON FE-3000 (anionic aqueous dispersion containing 50% of hydroxylated fluorinated polymer (particle size: 150 nm) by the company ASAHI GLASS.

5) silicone-based derivatives, such as that of the dispersion marketed under the reference HYCAR 26348 (acrylates/acrylonitrogen/siloxane copolymer) (particle size: 133 nm) by the company GOODRICH.

6) diisocyanate derivatives, such as that of the dispersion marketed under the reference NEOPAC E-106 (urethane/acrylate copolymer (and) methylpyrrolidone (and) triethylamine) (particle size: 98 nm) by the company AVECIA RESINS.

According to a preferred embodiment of the invention, particles of acrylic acid/ethyl acrylate copolymer, particles of styrene/butyl acrylate/methyl methacrylate/methacrylic acid hybrid copolymer, that is to say the dispersions sold under the names ACRYSOL 33 or ACULYN 33 by the company ROHM & HAAS and SETALUX 6801 AQ 24 by the company AKZO NOBEL, and mixtures thereof are used. When the polymer dispersion “ACULYN 33” is used, it is preferable that the composition has a pH value ranging from 3 to 6.3.

These dispersions are characterized by their calorimetric difference as indicated in the table below:

| | Appearance | Maximum | |
|---|---|---|---|
| | of the raw | colorimetric | Size of the |
| Raw material | material | difference | particles |
| ACULYN 33 | Iridescent | 12.5 | 110.2 ± 2.4% |
| | white milk | | |
| SETALUX 6801 | Iridescent | 11.1 | 167.7 ± 1.8% |
| AQ 24 | white milk | | |
| Comparative | Non- | Less than 1 | 252.2 ± 13.5% |
| example: | iridescent | | with two |
| SYNTHALEN | white milk | | populations of |
| W2000 ® of | | | sizes, one |
| 3V SA | | | centred around |
| | | | 167 nm, the |
| | | | other centred |
| | | | around 370 nm. |

As shown in this table, the particles of SYNTHALEN W2000 are not monodisperse, that is to say that their size is not homogeneous and comprises several Gaussian curves. The comparative results presented later show that this polymer dispersion is not appropriate for obtaining an iridescent effect, whereas the two dispersions “ACULYN 33” and “SETALUX 6801 AQ 24” which are monodisperse allow iridescent compositions to be obtained.

The quantity of polymer particles in the composition of the invention depends on the polymer used. It is preferably at least 3% by weight of active material relative to the total weight of the composition. It may range, for example, from 3 to 50%, preferably from 3.5 to 40% and even better from 4 to 30% by weight (of active material or dry matter) relative to the total weight of the composition. The weight of active material corresponds to the weight of 0.20 dry matter content of particles of polymers.

The composition according to the invention contains at least one cationic and/or amphoteric polymer. These cationic or amphoteric polymers possess a charge density of between 0.9 and 7 meq/g and preferably between 0.9 and 4 meq/g. They may be introduced at concentrations ranging from 0.01 to 5% by weight and preferably from 0.1 to 3% by weight of active material, relative to the total weight of the composition.

There may be used in the composition of the invention all the cationic or amphoteric polymers customarily used in the cosmetic or dermatological field.

There may be mentioned, for example, as cationic polymers which can be used in the composition of the invention, the polymers comprising at least one quaternary amine group and optionally primary, secondary, tertiary and/or quaternary amine groups which form part of the polymer chain or which are directly linked thereto. These polymers generally have a molecular weight ranging from 500 to about 5 000 000 and preferably from 1 000 to 3 000 000.

Among these polymers, there may be mentioned more particularly the following cationic polymers:

(1) The homopolymers or copolymers derived from acrylic or methacrylic esters or amides and comprising at least one quaternary ammonium group, and in particular one of the following units of formulae:

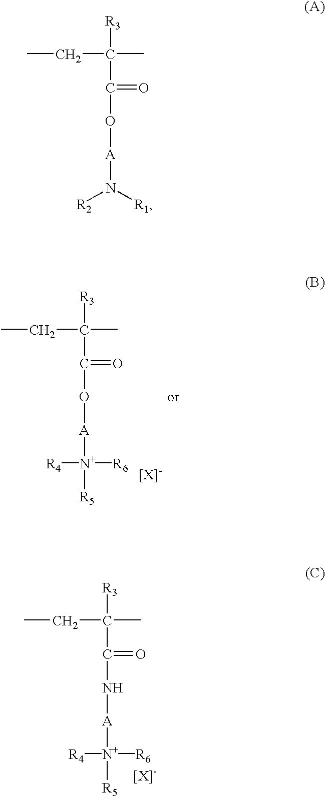

in which:

R₃ denotes a hydrogen atom or a CH₃ radical;

A is a linear or branched alkyl group of 1 to 6 carbon atoms or a hydroxyalkyl group comprising from 1 to 4 carbon atoms;

R₄, R₅, R₆, which are identical or different, represent an alkyl group having from 1 to 18 carbon atoms or a benzyl radical;

R₁ and R₂ represent a hydrogen atom or an alkyl group having from 1 to 6 carbon atoms;

X denotes a methosulphate anion or a halide such as chloride or bromide.

The copolymers of the family (1) contain, in addition, one or more units derived from comonomers which may be chosen from the family of acrylamides, methacrylamides, diacetone acrylamides, acrylamides and methacrylamides substituted on the nitrogen with lower alkyls, acrylic or methacrylic acids or esters thereof, vinyllactams such as vinylpyrrolidone or vinylcaprolactam, vinyl esters.

Thus, among these copolymers of the family (1), there may be mentioned for example:

the copolymers of acrylamide and dimethylaminoethyl methacrylate quaternized with dimethyl sulphate or with a dimethyl halide, such as the product marketed under the name HERCOFLOC by the company HERCULES;

the copolymers of acrylamide and methacryloyloxyethyltrimethylammonium chloride, such as POLYQUATERNIUM 5 (CTFA name) and for example the product marketed under the name MERQUAT 5 by the company CALGON; and such as POLYQUATERNIUM 15 (CTFA name) and for example the product marketed under the name ROHAGIT KF 720 F by the company ROHM;

the copolymer of acrylamide and methacryloyloxyethyltrimethylammonium methosulphate marketed under the name RETEN by the company HERCULES;

the vinylpyrrolidone/dialkylaminoalkyl acrylate or methacrylate copolymers, quaternized or otherwise, such as POLYQUATERNIUM 11 (CTFA name) and for example the products marketed under the names GAFQUAT 755, GAFQUAT 755N and GAFQUAT 734 by the company ISP;

the dimethylaminoethyl/vinylcaprolactam/vinylpyrrolidone terpolymers such as the product sold under the name GAFFIX VC 713 by the company ISP;

the quaternized vinylpyrrolidone/dimethylaminopropyl methacrylamide copolymer such as POLYQUATERNIUM 28 (CTFA name) and for example the product marketed under the name GAFQUAT HS-100 by the company ISP;

the copolymers based on vinylpyrrolidone and vinylcaprolactam such as POLYQUATERNIUM 46 (CTFA name) and for example the product marketed under the name LUVIQUAT HOLD by the company BASF;

(2) The homopolymers or copolymers of dimethyldiallylammonium of formula (2) described below:

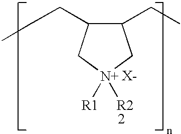

in which:

R1 and R2, which are identical or different, denote a hydrogen atom, or represent an alkyl group having from 1 to 18 carbon atoms;

X denotes a methosulphate anion or a halide such as chloride or bromide.

The copolymers of the family (2) contain, in addition, one or more units derived from comonomers which may be chosen from the family of acrylamides, methacrylamides, diacetone acrylamides, acrylamides and methacrylamides substituted on the nitrogen with lower alkyl (C1-C6) radicals, acrylic or methacrylic acids or esters thereof, vinyllactams such as vinylpyrrolidone or vinylcaprolactam, vinyl esters.

As polymers of the family (2), there may be mentioned, for example, the following polymers:

the polymers of dimethyldiallylammonium chloride, such as POLYQUATERNIUM 6 (CTFA name) and for example the products marketed under the names SALCARE SC 310 by the company CIBA, and MERQUAT 100 by the company CALGON;

the copolymers of acrylamide and dimethyldiallylammonium chloride such as POLYQUATERNIUM 7 (CTFA name) and for example the products marketed under the names MERQUAT S, MERQUAT 2200 and MERQUAT 550 by the company CALGON, SALCARE SC 10 by the company CIBA.

(3) The quaternized polysaccharides such as the guar gums containing cationic trialkylammonium groups, such as the products marketed in particular under the trade names JAGUAR C 13 S, JAGUAR C 15, JAGUAR C 17, JAGUAR C 162, JAGUAR C 2000, JAGUAR EXCEL by the company MEYHALL; or such as the quaternized cellulose derivatives, such as the hydroxyethyl cellulose polymer containing cationic triallylammonium groups, such as POLYQUATERNIUM 10 (CTFA name) and for example the product marketed under the name UCARE POLYMER JR-400 by the company AMERCHOL.

(4) The quaternary copolymers of vinylpyrrolidone and vinylimidazole; such as POLYQUATERNIUM 16 (CTFA name) and for example the products marketed under the names LUVIQUAT FC905, LUVIQUAT FC370, LUVIQUAT HM552 and LUVIQUAT FC550 by the company BASF and POLYQUATERNIUM 44 (CTFA name) and for example the product marketed under the name LUVIQUAT CARE by the company BASF.

(5) Chitosans or their salts such as chitosan acetate, lactate, glutamate, gluconate or pyrrolidonecarboxylate. Among these compounds, there may be mentioned the chitosan having a deacetylation level of 90.5% by weight sold under the name KYTAN BRUT STANDARD by the company ABER TECHNOLOGIES, chitosan pyrrolidonecarboxylate sold under the name KYTAMER PC by the company AMERCHOL.

(6) Cationic cellulose derivatives such as cellulose copolymers or cellulose derivatives grafted with a water-soluble monomer comprising a quaternary ammonium, and described in particular in U.S. Pat. No. 4,131,576, such as hydroxyalkyl celluloses like hydroxymethyl, hydroxyethyl or hydroxypropyl celluloses grafted in particular with a methacryloyloxyethyltrimethylammonium, methacrylamidopropyltrimethylammonium or dimethyldiallylammonium salt. The marketed products corresponding to this definition are more particularly the products sold under the name “CELQUAT L 200” and “CELQUAT H 100” by the company National Starch, and mixtures of these cationic polymers.

The polymers which are particularly preferred are POLYQUATERNIUM 5, POLYQUATERNIUM 7, POLYQUATERNIUM 28, POLYQUATERNIUM 39, POLYQUATERNIUM 44 and POLYQUATERNIUM 47 because they impart a great deal of smoothness on the finished product.

As amphoteric polymers which can be used in the composition, there may be mentioned the polymers comprising K and M units randomly distributed in the polymer chain where K denotes a unit derived from a monomer comprising at least one basic nitrogen atom and M denotes a unit derived from an acidic monomer comprising one or more carboxylic or sulphonic groups, or K and M may denote groups derived from zwitterionic monomers of carboxybetaines or sulphobetaines; K and M may also denote a cationic polymer chain comprising primary, secondary, tertiary or quaternary amine groups, in which at least one of the amine groups carries a carboxylic or sulphonic group linked via a hydrocarbon radical, or K and M form part of a chain of a polymer with an α,β-dicarboxylic ethylene unit in which one of the carboxylic groups has been caused to react with a polyamine comprising one or more primary or secondary amine groups.

The amphoteric polymers which are more particularly preferred are chosen from the following polymers:

(1) the polymers resulting from the copolymerization of a monomer derived from a vinyl compound carrying a carboxylic group such as more particularly acrylic acid, methacrylic acid, maleic acid, alpha-chloroacrylic acid, and of a basic monomer derived from a substituted vinyl compound containing at least one basic atom such as more particularly dialkylaminoalkyl methacrylate and acrylate, dialkylaminoalkyl methacrylamide and acrylamide. There may be mentioned, for example, the sodium acrylate/acrylamidopropyltrimethylammonium chloride copolymer sold under the name POLYQUART KE 3033 by the company HENKEL, the acrylic acid/methacrylate/methacrylamidopropyltrimonium chloride terpolymers, such as POLYQUATERNIUM 47 (CTFA name) and for example the product marketed under the name MERQUAT 2001 by the company CALGON.

The vinyl compound may also be a dialkyldiallylammonium salt such as diethyldiallylammonium chloride. The copolymers of acrylic acid and of the latter monomer (POLYQUATERNIUM-22) are for example those marketed under the names MERQUAT 280, MERQUAT 295 and MERQUAT PLUS 3330 by the company CALGON.

(2) The polymers comprising units derived from:

a) at least one monomer chosen from acrylamides or methacrylamides substituted on the nitrogen with an alkyl radical, in particular the N-alkyl-substituted acrylamides or methacrylamides in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

b) at least one acidic comonomer containing one or more reactive carboxylic groups, such as acrylic, methacrylic, crotonic, itaconic, maleic and fumaric acids and the alkyl monoesters having 1 to 4 carbon atoms of maleic or fumaric anhydrides or acids; and

c) at least one basic comonomer such as esters with primary, secondary, tertiary and quaternary amine substituents of acrylic and methacrylic acids and the quaternization product of dimethylaminoethyl methacrylate with dimethyl or diethyl sulphate. The preferred basic comonomers are methacrylates of aminoethyl, butylaminoethyl, N,N′-dimethylaminoethyl and N-tert-butylaminoethyl.

As polymers comprising these units, there may be mentioned “Octylacrylamide/acrylates/butylaminoethylmethacrylate copolymer” (CTFA name) such as the products sold under the name AMPHOMER or LOVOCRYL 47 by the company NATIONAL STARCH.

(3) The crosslinked and alkylated (partially or completely) polyaminoamides which are crosslinked by addition of a bifunctional crosslinking agent chosen from epihalohydrins, diepoxides, dianhydrides, bis-unsaturated derivatives, using 0.025 to 0.35 mol of crosslinking agent per amine group of the polyaminoamide, and alkylated by the action of acrylic acid, chloroacetic acid or an alkane sultone or their salts.

(4) The polymers comprising zwitterionic units of formula (I):

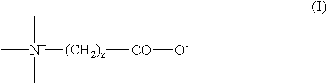

The polymers containing such units may also comprise units derived from nonzwitterionic monomers such as dimethyl or diethylaminoethyl acrylate or methacrylate or alkylacrylates or methacrylates, acrylamides or methacrylamides or vinyl acetate.

By way of example, there may be mentioned the copolymer of methyl methacrylate/dimethylcarboxymethylammoniumethyl methacrylate such as the product sold under the name DIAFORMER Z301 by the company SANDOZ.

(5) The amphoteric polymers derived from chitosan such as the polymers derived from the N-carboxyalkylation of chitosan such as N-carboxymethylchitosan or N-carboxybutylchitosan sold under the name EVALSAN by the company JAN DEKKER.

(6) Amphoteric polymers derived from chloroacetic acid or sodium chloroacetate.

(7) The copolymers (C₁-C₅)alkyl vinyl ether/maleic anhydride partially modified by semiamidation with an N,N-dialkylaminoalkylamine such as N,N-dimethylaminopropylamine or by semiesterification with an N,N-dialkanolamine. These copolymers may also comprise other vinyl comonomers such as vinylcaprolactam, and mixtures of these amphoteric polymers.

The amphoteric polymers which are particularly preferred according to the invention are acrylic acid/methacrylate/methacrylamidopropyltrimonium chloride terpolymers, such as POLYQUATERNIUM 47 (CTFA name) and for example the product marketed under the name MERQUAT 2001 by the company CALGON, MERQUAT 280, MERQUAT 295 and MERQUAT PLUS 3330 by the company CALGON.

The physiologically acceptable medium of the composition of the invention comprises water. The quantity of water may range from 30 to 96.99% by weight and preferably from 40 to 95% by weight relative to the total weight of the composition. This medium may contain, in addition to water, one or more solvents chosen from lower alcohols comprising from 1 to 8 carbon atoms, such as ethanol; polyols such as glycerin; glycols such as butylene glycol; isoprene glycol, propylene glycol, polyethylene glycols such as PEG-8; sorbitol; sugars such as glucose, fructose, maltose, lactose, sucrose; and mixtures thereof. The quantity of solvent(s) in the composition of the invention may range from 0.5 to 30% by weight and preferably from 5 to 20% by weight relative to the total weight of the composition.

The composition of the invention may also contain one or more additives that are customary in the cosmetic field, such as mattifying inorganic or organic fillers, surfactants, active agents, preservatives, gelling agents, plasticizers, antioxidants, perfumes, odour absorbers, antifoaming agents, sequestrants (EDTA), acidic or basic pH regulators, or buffers, pigments and pearlescent agents (this addition only being made if it does not disrupt the desired effect), fatty substances made compatible with the aqueous medium, such as oils or waxes, and mixtures thereof, insofar as the additive does not impair the properties sought for the composition of the invention. The quantities of these various additives are those conventionally used in the fields considered, and for example from 0.01 to 20% of the total weight of the composition.

As surfactants, there may be used any nonionic or ionic (anionic or cationic) surfactant. 1) As nonionic surfactants, there may be mentioned, for example, alkyl polyglycosides (APG), esters of polyols and of fatty acids, esters of polyethylene glycols and of fatty acid, derivatives of fatty alcohols and of polyols (ethers), and oxyalkylenated (oxyethylenated and/or oxypropylenated) derivatives of these compounds. There may also be mentioned maltose esters, polyglycerolated fatty alcohols, glucamine derivatives such as 2-ethylhexyloxycarbonyl-n-methylglucamine, and mixtures thereof.

As alkyl polyglucosides, there may be mentioned, for example, decyl glucoside (C9/C11 alkyl polyglucoside (1.4)) such as the product marketed under the name MYDOL 10 by the company Kao Chemicals, the product marketed under the name PLANTAREN 2000 UP and PLANTACARE 2000 UP by the company Henkel, and the product marketed under the name ORAMIX NS 10 by the company Seppic; caprylyl/capryl glucoside such as the product marketed under the name ORAMIX CG 110 by the company Seppic or under the name LUTENSOL GD 70 by the company BASF; lauryl glucoside such as the products marketed under the names PLANTAREN 1200 N and PLANTACARE 1200 by the company Henkel; and cocoglucoside such as the product marketed under the name PLANTACARE 818/UP by the company Henkel, and mixtures thereof.

The maltose derivatives are, for example, those disclosed in document EP-A-566 438, such as o-octanoyl-6′-D-maltose or o-dodecanoyl-6′-D-maltose disclosed in document FR-2 739 556.

Among the polyglycerolated fatty alcohols which may be mentioned are polyglycerolated dodecanediol (3.5 mol of glycerol), this product being sold under the name CHIMEXANE NF by the company Chimex. 2) Anionic surfactants which may be used, for example, include carboxylates, amino acid derivatives, alkyl sulphates, alkyl ether sulphates, sulphonates, isethionates, taurates, sulphosuccinates, alkyl sulphoacetates, phosphates and alkyl phosphates, polypeptides, anionic alkyl polyglucoside derivatives and fatty acid soaps, and mixtures thereof.

Carboxylates which may be mentioned, for example, include alkali metal salts of N-acylamino acids; amido ether carboxylates (AECs), for instance sodium lauryl amido ether carboxylate (3 EO) sold under the name AKYPO FOAM 30 by the company Kao Chemicals; polyoxyethylenated carboxylic acid salts, for instance oxyethylenated (6 EO) sodium lauryl ether carboxylate (C12-14-16 65/25/10) sold under the name AKYPO SOFT 45 NV by the company Kao Chemicals; polyoxyethylenated fatty acids of olive oil and of carboxymethyl, for instance the product being sold under the name OLIVEM 400 by the company Biologia E Tecnologia; oxyethylenated (6 EO) sodium tridecyl ether carboxylate sold under the name NIKKOL ECTD-6NEX by the company NIKKOL and sodium 2-(2-hydroxy-alkyloxy)acetate sold under the name BEAULIGHT SHAA by the company SANYO.

The amino acid derivatives may be chosen, for example, from sarcosinates and in particular acylsarcosinates, for instance the sodium lauroyl sarcosinate sold under the name SARKOSYL NL 97 by the company Ciba or sold under the name ORAMIX L 30 by the company Seppic, the sodium myristoyl sarcosinate sold under the name NIKKOL SARCOSINATE MN by the company Nikkol or the sodium palmitoyl sarcosinate sold under the name NIKKOL SARCOSINATE PN by the company Nikkol; alaninates, for instance the sodium N-lauroyl-N-methylamidopropionate sold under the name SODIUM NIKKOL ALANINATE LN 30 by the company Nikkol or sold under the name ALANONE ALE by the company Kawaken, and the N-lauroyl-N-methylalanine triethanolamine sold under the name ALANONE ALTA by the company Kawaken; N-acylglutamates, for instance the triethanolamine monococoylglutamate sold under the name ACYLGLUTAMATE CT-12 by the company Ajinomoto and the triethanolamine lauroylglutamate sold under the name ACYLGLUTAMATE LT-12 by the company Ajinomoto; aspartates, for instance the mixture of triethanolamine N-lauroyl aspartate and of triethanolamine N-myristoylaspartate, sold under the name ASPARACK LM-TS2 by the company Mitsubishi; citrates, and mixtures thereof.

Glycine derivatives which may be mentioned include sodium N-cocoylglycinate and potassium N-cocoylglycinate, for instance the products sold under the names AMILITE GCS-12 and AMILITE GCK-12 by the company Ajinomoto.

Alkyl ether sulphates which may be mentioned, for example, include the sodium lauryl ether sulphate (C12-14 70/30) (2.2 EO) sold under the names SIPON AOS 225 or TEXAPON N702 PATE by the company Henkel, the ammonium lauryl ether sulphate (C12-14 70/30) (3 EO) sold under the name SIPON LEA 370 by the company Henkel, and the ammonium (C12-C14) alkyl ether (9 EO) sulphate sold under the name RHODAPEX AB/20 by the company Rhodia Chimie.

Sulphonates which may be mentioned, for example, include alpha-olefin sulphonates, for instance the sodium alpha-olefin sulphonate (C14-16) sold under the name BIO-TERGE AS-40 by the company Stepan, sold under the names WITCONATE AOS PROTEGE and SULFRAMINE AOS PH 12 by the company Witco or sold under the name BIO-TERGE AS-40 CG by the company Stepan, the sodium secondary olefin sulphonate sold under the name HOSTAPUR SAS 30 by the company Clariant; linear alkyl aryl sulphonates, for instance the sodium xylene sulphonate sold under the names MANROSOL SXS30, MANROSOL SXS40 and MANROSOL SXS93 by the company Manro.

Isethionates which may be mentioned include acylisethionates, for instance sodium cocoylisethionate, such as the product sold under the name JORDAPON CI P by the company Jordan.

Taurates which may be mentioned include the sodium salt of palm kernel oil methyltaurate sold under the name HOSTAPON CT PATE by the company Clariant; N-acyl N-methyltaurates, for instance the sodium N-cocoyl N-methyltaurate sold under the name HOSTAPON LT-SF by the company Clariant or sold under the name NIKKOL CMT-30-T by the company Nikkol, and the sodium palmitoyl methyltaurate sold under the name NIKKOL PMT by the company Nikkol.

Sulphosuccinates which may be mentioned, for example, include the oxyethylenated (3 EO) lauryl alcohol monosulphosuccinate (C12/C14 70/30) sold under the names SETACIN 103 SPECIAL, REWOPOL SB-FA 30 K 4 by the company Witco, the disodium salt of a C12-C14 alkyl hemisulphosuccinate, sold under the name SETACIN F SPECIAL PASTE by the company Zschimmer Schwarz, the oxyethylenated (2 EO) disodium oleamidosulphosuccinate sold under the name STANDAPOL SH 135 by the company Henkel, the oxyethylenated (5 EO) laurylamide monosulphosuccinate sold under the name LEBON A-5000 by the company Sanyo, the oxyethylenated (10 EO) disodium salt of lauryl citrate monosulphosuccinate sold under the name REWOPOL SB CS 50 by the company Witco, and the ricinoleic monoethanolamide monosulphosuccinate sold under the name REWODERM S 1333 by the company Witco.

Phosphates and alkyl phosphates which may be mentioned, for example, include monoalkyl phosphates and dialkyl phosphates, such as the lauryl monophosphate sold under the name MAP 20 by the company Kao Chemicals, the potassium salt of dodecylphosphoric acid, as a mixture of monoester and diester (mainly diester), sold under the name CRAFOL AP-31 by the company Cognis, the mixture of monoester and diester of octylphosphoric acid, sold under the name CRAFOL AP-20 by the company Cognis, the mixture of ethoxylated (7 mol of EO) phosphoric acid monoester and diester of 2-butyloctanol, sold under the name ISOFOL 12 7 EO-PHOSPHATE ESTER by the company Condea, the potassium salt or triethanolamine salt of monoalkyl (C12-C13) phosphate sold under the references ARLATONE MAP 230K-40 and ARLATONE MAP 230T-60 by the company Uniqema, and the potassium lauryl phosphate sold under the name DERMALCARE MAP XC-99/09 by the company Rhodia Chimie.

The polypeptides are obtained, for example, by condensing a fatty chain with amino acids from cereals and in particular from wheat and oat. Polypeptides which may be mentioned, for example, include the potassium salt of hydrolysed lauroyl wheat protein, sold under the name AMINOFOAM W OR by the company Croda, the triethanolamine salt of hydrolysed cocoyl soybean protein, sold under the name MAY-TEIN SY by the company Maybrook, the sodium salt of oat lauroyl amino acids, sold under the name PROTEOL OAT by the company SEPPIC, the collagen hydrolysate grafted onto coconut fatty acid, sold under the name GELIDERM 3000 by the company Deutsche Gelatine, and the soybean proteins acylated with hydrogenated coconut acids, sold under the name PROTEOL VS 22 by the company Seppic.

The anionic derivatives of alkyl polyglucosides may be, in particular, glyceryl ethers, carbonates, sulphosuccinates, tartrates and citrates obtained from alkyl polyglucosides. Mention may be made, for example, of the sodium salt of cocoylpolyglucoside (1,4) tartaric ester, sold under the name EUCAROL AGE-ET by the company Cesalpinia, the disodium salt of cocoylpolyglucoside (1,4) sulphosuccinic ester, sold under the name ESSAI 512 MP by the company Seppic, and the sodium salt of cocoylpolyglucoside (1,4) citric ester, sold under the name EUCAROL AGE-EC® by the company Cesalpinia. Another anionic saccharide derivative may be sodium dodecyl-D-galactoside uronate sold under the name DODECYL-D-GALACTOSIDE URONATE DE SODIUM by the company SOLIANCE.

The fatty acid soaps which may be used as anionic surfactants are fatty acids of natural or synthetic origin, salified with a mineral or organic base. The fatty chain may comprise from 6 to 22 carbon atoms and preferably from 8 to 18 carbon atoms. The mineral or organic base may be chosen from alkali metals or alkaline-earth metals, amino acids and amino alcohols. Salts which may be used, for example, include the sodium, potassium, magnesium, triethanolamine and N-methylglucamine salts of lysine and of arginine. Soaps which may be mentioned, for example, include the potassium or sodium salts of lauric, myristic, palmitic or stearic acid (potassium or sodium laurate, myristate, palmitate and stearate), and mixtures thereof.

3) The amphoteric and zwitterionic surfactants which may be used, for example, include betaines, N-alkylamidobetaines and derivatives thereof, glycine derivatives, sultaines, alkyl polyaminocarboxylates and alkylamphoacetates, and mixtures thereof.

Betaines which may be mentioned, for example, include cocobetaine, for instance the product sold under the name DEHYTON AB-30 by the company Henkel, laurylbetaine, for instance the product sold under the name GENAGEN KB by the company Clariant, oxyethylenated (10 EO) laurylbetaine, for instance the product sold under the name LAURYL ETHER (10 EO) BETAINE by the company Shin Nihon Rica, and oxyethylenated (10 EO) stearylbetaine, for instance the product sold under the name STEARYL ETHER (10 EO) BETAINE by the company Shin Nihon Rica.

Among the N-alkylamidobetaines and derivatives thereof which may be mentioned, for example, are the cocamidopropylbetaine sold under the name LEBON 2000 HG by the company Sanyo, or sold under the name EMPIGEN BB by the company Albright & Wilson, and the lauramidopropyl betaine sold under the name REWOTERIC AMB12P by the company Witco.

Sultaines which may be mentioned include the cocoylamidopropylhydroxysulphobetaine sold under the name CROSULTAINE C-50 by the company Croda.

Alkyl polyaminocarboxylates (APACS) which may be mentioned include the sodium cocoylpolyaminocarboxylate sold under the name AMPHOLAK 7 CX/C and AMPHOLAK 7 CX by the company Akzo Nobel, the sodium stearylpolyamidocarboxylate sold under the name AMPHOLAK 7 TX/C by the company Akzo Nobel and the sodium carboxymethyloleylpolypropylamine sold under the name AMPHOLAK X07/C by the company Akzo Nobel.

Alkylamphoacetates which may be mentioned, for example, include N-disodium N-cocoyl-N-carboxymethoxyethyl-N-carboxymethylethylenediamine (CTFA name: disodium cocamphodiacetate), for instance the product sold under the name MIRANOL C2M CONCENTRE NP by the company Rhodia Chimie and N-sodium N-cocoyl-N-hydroxyethyl-N-carboxymethylethylenediamine (CTFA name: sodium cocamphoacetate).

According to a preferred embodiment of the invention, the composition comprises at least one nonionic surfactant, and in particular one alkyl polyglycoside.

When one or more surfactants are present in the composition of the invention, the quantity of surfactant may range, for example, from 0.01 to 40% by weight of active material and preferably from 0.5 to 30% by weight of active material relative to the total weight of the composition.

As active agents which can be used in the composition of the invention, there may be mentioned any active agent normally used in the cosmetic and dermatological fields, such as water-soluble or fat-soluble vitamins or provitamins, for example vitamins A (retinol), C (ascorbic acid), B3 or PP (niacinamide), B5 (panthenol), E (tocopherol), K1, beta-carotene. and the derivatives of these vitamins and in particular their esters; hormones or derivatives such as DHEA and 7α-hydroxy DHEA; antiseptics; antiseborrhoeics; antimicrobials such as benzoyl peroxide, salicylic acid, triclosan, azelaic acid, niacinamide (vit. PP); moisturizers such as glycerin, hyaluronic acid, pyrrolidone carboxylic acid (PCA) and its salts, sodium pidolate, serine, xylitol, trehalose, ectoine, ceramides, urea; keratolytic and antiageing agents such as alpha-hydroxy acids such as glycolic acid, citric acid, lactic acid, beta-hydroxy acids such as salicylic acid, coenzyme Q10; sunscreens; optical brighteners; slimming agents such as caffeine, theophylline, theobromine, anti-inflammatory agents such as P-glycyrrhetinic acid and ursolic acid. It is also possible to use a mixture of two or more of these active agents. The active agent(s) may be, for example, present in a concentration ranging from 0.01 to 20%, preferably from 0.1 to 10% and even better from 0.5 to 5% of the total weight of the composition.

The composition of the invention may be provided in any of the galenic forms normally used in the cosmetic and dermatological fields. It is generally provided in the form of a gel and is prepared according to the customary methods, that is to say by mixing the polymer dispersion and the cationic and/or amphoteric polymer in the aqueous medium and optionally adjusting the pH.

The composition according to the invention may comprise an oily phase (or fatty phase) comprising at least one oil, provided that it is solubilized in the medium, for example by forming a microemulsion. The oil(s) may be chosen from oils of plant origin (jojoba, avocado, sesame, sunflower, maize, soyabean, safflower, grapeseed), mineral oils (petroleum jelly, hydrogenated or nonhydrogenated isoparaffins, isohexadecane, squalane), synthetic oils (parleam, isopropyl myristate, cetearyl octanoate, polyisobutylene, ethylhexyl palmitate, alkyl benzoates), volatile or nonvolatile silicone oils such as polydimethylsiloxanes (PDMS) and cyclodimethylsiloxanes or cyclomethicones, and fluorinated or fluorosilicone oils, as well as mixtures of these oils. The oily phase may contain, in addition, other fatty constituents such as fatty alcohols such as stearyl alcohol, cetyl alcohol and the mixture thereof (cetearyl alcohol); fatty acids; waxes; silicone gums. The oily phase may be present in a quantity ranging from 0.01 to 20% by weight relative to the total weight of the composition.

The composition according to the invention finds application in a large number of treatments, in particular cosmetic treatments, of the skin, including the scalp, the hair, the nails and/or the mucous membranes, in particular for the care, cleansing and/or application of make-up to and/or sun-protection of the skin, the hair and/or the lips or the mucous membranes. The composition according to the invention also makes it possible to attach the active agents to the surface of the skin. It is possible to use it, for example, as cleansing or make-up-removing products for the face (including the eyes and the lips) or the body, as care products for the face, for example for treating the signs of ageing or for moisturizing or nourishing the skin, as anti-sun products for protecting the skin or the hair from UV radiation, as hair products for washing or protecting the hair, for example in the form of shampoos, sleeking products or lacquers.

Accordingly, the subject of the present invention is the cosmetic use of the composition as defined above, for the treatment, protection, care, removal of make-up from and/or cleansing of the skin, the lips and/or the hair, and/or for the application of make-up to the skin and/or the lips.

The subject of the present invention is also a method for the cosmetic treatment of the skin, including the scalp, the hair and/or the lips, characterized in that a cosmetic composition as defined above is applied to the skin, the hair and/or the lips.

The following examples illustrate the invention. A.M. means therein “as active material”. The quantities are indicated as a percentage by weight unless otherwise stated.

### EXAMPLE 1

### Composition for Cleansing the Face and the Body

| | | |
|---|---|---|
| ACULYN 33 ® from ROHM-HAAS | 5 | % A.M. |
| Decyl glucoside (Plantacare 2000 UP | 13 | % A.M. |
| from COGNIS, containing 55% of A.M. | | |
| in water) | | |
| POLYQUATERNIUM-47 (MERQUAT 2001 ® from | 0.5 | % A.M. |
| CALGON) | | |
| Glycerin | 3 | % |
| Preservatives | qs | |
| Sodium hydroxide at 10% | qs pH | |
| Water | qs 100 | % |

Procedure: the water, the glycerin, the surfactant and the preservatives are mixed at room temperature (about 20 to 25° C.). Aculyn 33 is added and the mixture is homogenized, and then the pH is adjusted to 5.80 by addition of sodium hydroxide at 10%. Finally, the cationic polymer is added and the mixture is homogenized.

An iridescent translucent gel of pH 5.80 is obtained whose colours vary from blue, green, yellow, to orange depending on the orientation. This gel has a viscosity, after 10 minutes, measured with rotor 3 or 4 (Rheomat RM180 at room temperature, that is to say at about 25° C., at 200 s⁻¹), of 12 poises (1.2 Pa.s), and it has a conductance, measured by means of the CD78 type electronic conductimeter from TACUSSEL at about 25° C., of 5410 μΩ⁻¹.

### EXAMPLE 2

### Composition for Cleansing the Face and the Body

| | | |
|---|---|---|
| ACULYN 33 ® from ROHM-HAAS | 5 | % A.M. |
| Decyl glucoside (Plantacare 2000 UP | | |
| from COGNIS, containing 55% of A.M. | | |
| in water) | 13 | % A.M. |
| POLYQUATERNIUM-5 (MERQUAT 5 ® from | | |
| NALCO) | 0.5 | % A.M. |
| Glycerin | 3 | % |
| Preservatives | qs | |
| Sodium hydroxide at 10% | qs pH | |
| Water | qs 100 | % |

The procedure is the same as for Example 1.

An iridescent translucent gel of pH 5.85 is obtained whose colours vary from blue, green, yellow, to orange depending on the orientation. This gel has a viscosity, after 10 minutes, measured with rotor 3 or 4 (Rheomat RM180 at room temperature, that is to say at about 25° C., at 200 s⁻¹), of 14 poises (1.4 Pa.s), and it has a conductance, measured by means of the CD78 type electronic conductimeter from TACUSSEL at about 25° C., of 4886 μΩ⁻¹.

1. Iridescent composition for topical application, comprising at least one polymer chosen from cationic polymers and amphoteric polymers, and monodisperse particles of polymer in aqueous dispersion, the said particles having a mean size in numerical terms ranging from 50 to 300 nm.

2. Composition according to the preceding claim, characterized in that it exhibits a turbidity of greater than 100.

3. Composition according to either of the preceding claims, characterized in that the said composition and/or the dispersion of particles possess a calorimetric difference ranging from 2 to 100.

4. Composition according to any one of the preceding claims, characterized in that it has a viscosity ranging from 0.1 Pa.s to 10 Pa.s.

5. Composition according to any one of the preceding claims, characterized in that the particles comprise polymer obtained from one or more monomers chosen from styrene, butadiene, ethylene, acrylonitrile, chloroprene, vinylidene chloride, isoprene, isobutylene and vinyl chloride, acrylic, methacrylic, vinylacetic, maleic, crotonic and itaconic acids and esters thereof, and mixtures thereof.

6. Composition according to any one of the preceding claims, characterized in that the particles comprise anionic polymer.

7. Composition according to the preceding claim, characterized in that the particles comprise homopolymers of acrylic acid, copolymers of acrylic acid, styrene derivatives, fluorinated derivatives, silicone-based derivatives, diisocyanate derivatives.

8. Composition according to any one of the preceding claims, characterized in that the polymer particles are chosen from particles of acrylic acid/ethyl acrylate copolymer, particles of styrene/butyl acrylate/methyl methacrylate/methacrylic acid hybrid copolymer, and mixtures thereof.

9. Composition according to any one of the preceding claims, characterized in that the quantity of polymer particles is at least 3% by weight of active material relative to the total weight of the composition.

10. Composition according to any one of the preceding claims, characterized in that the quantity of polymer particles ranges from 3 to 50% and preferably from 3.5 to 40% by weight of active material relative to the total weight of the composition.

11. Composition according to any one of the preceding claims, characterized in that the cationic polymer is chosen from homopolymers or copolymers derived from acrylic or methacrylic esters or amides and comprising at least one quaternary ammonium group, quaternized polysaccharides, quaternary copolymers of vinylpyrrolidone and of vinylimidazole, chitosans or their salts, cationic cellulose derivatives and mixtures thereof.

12. Composition according to the preceding claim, characterized in that the cationic polymer is chosen from Polyquaternium 5, Polyquaternium 6, Polyquaternium 7, Polyquaternium 10, Polyquaternium 11, Polyquaternium 15, Polyquaternium 16, Polyquaternium 28, Polyquaternium 39, Polyquaternium 44, Polyquaternium 46, and mixtures thereof.

13. Composition according to any one of the preceding claims, characterized in that the amphoteric polymer is chosen from polymers resulting from the copolymerization of a monomer derived from a vinyl compound carrying a carboxylic group; polymers comprising units derived from at least one monomer chosen from acrylamides or methacrylamides substituted on the nitrogen with an alkyl radical, from at least one acidic comonomer containing one or more reactive carboxylic groups and from at least one basic comonomer; crosslinked and alkylated polyaminoamides; polymers comprising zwitterionic units; polymers derived from chitosan; polymers derived from the N-carboxyalkylation of chitosan, Polyquaternium 22, Polyquaternium 47, and mixtures thereof.

14. Composition according to the preceding claim, characterized in that the amphoteric polymer is chosen from acrylic acid/methacrylate/methacrylamidopropyltrimonium chloride terpolymers.

15. Composition according to any one of the preceding claims, characterized in that the quantity of cationic and/or amphoteric polymer ranges from 0.01 to 5% by weight relative to the total weight of the composition.

16. Composition according to any one of the preceding claims, characterized in that it contains, in addition, at least one additive chosen from fillers, surfactants, active agents, preservatives, gelling agents, plasticizers, antioxidants, perfumes, odour absorbers, antifoaming agents, sequestrants, pH regulators, buffers, pigments and pearlescent agents, fatty substances.

17. Composition according to any one of the preceding claims, characterized in that it contains at least one surfactant.

18. Composition according to the preceding claim, characterized in that the surfactant is a nonionic surfactant.

19. Composition according to claim 17 or 18, characterized in that the quantity of surfactant(s) ranges from 0.01 to 40% by weight relative to the total weight of the composition.

20. Composition according to any one of the preceding claims, characterized in that it comprises 30 to 96.99% by weight of water relative to the total weight of the composition.

21. Composition according to any one of the preceding claims, characterized in that it comprises one or more solvents chosen from alcohols comprising from 1 to 8 carbon atoms, polyols, sugars and mixtures thereof.

22. Composition according to any one of the preceding claims, characterized in that it constitutes a cosmetic composition.

23. Cosmetic use of the composition according to any one of claims 1 to 22, for the treatment, protection, care, removal of make-up from and/or cleansing of the skin, the lips and/or the hair and/or for the application of make-up to the skin and/or the lips.

24. Method for the cosmetic treatment of the skin, including the scalp, the hair, and/or the lips, characterized in that a cosmetic composition according to any one of claims 1 to 22 is applied to the skin, the hair and/or the lips.
